(19) United States
(12) Patent Application Publication
(10) Pub. No.: US 2024/0390228 A1
(43) Pub. Date: Nov. 28, 2024

(54) SEALING MEMBER AND BLOOD BAG SET

(71) Applicant: TERUMO KABUSHIKI KAISHA, Tokyo (JP)

(72) Inventor: Ryoji KATAOKA, Fujinomiya-shi Shizuoka (JP)

(21) Appl. No.: 18/794,905

(22) Filed: Aug. 5, 2024

Related U.S. Application Data
(63) Continuation of application No. PCT/JP2023/008443, filed on Mar. 7, 2023.

(30) Foreign Application Priority Data
Mar. 7, 2022 (JP) 2022-034092

(51) Int. Cl. A61J 1/12; A61J 1/14

(52) U.S. Cl. CPC A61J 1/12; A61J 1/14

(57) ABSTRACT

A sealing member for occluding a medical tube of a blood bag set includes a base for contact with an inner face of the medical tube, the base having a base flow path inside the base; a plug located at a distal end of the base, the plug occluding the base flow path; and a breakable portion including a groove that is a thinnest portion extending in a circumferential direction along the boundary between the proximal end of the plug and the base, in which, in viewing from a side of location of a distal end of the sealing member in a direction of an axis of the sealing member, the breakable portion is non-circular in shape, the breakable portion including a plurality of tops.

### CROSS-REFERENCE TO RELATED APPLICATIONS

The present disclosure is a continuation of and claims benefit to PCT/JP2023/008443 filed on Mar. 7, 2023, entitled “SEALING MEMBER AND BLOOD BAG SET” which claims priority to Japanese Patent Application No. 2022-034092 filed on Mar. 7, 2022. The entire disclosure of the applications listed above are hereby incorporated herein by reference, in their entireties, for all that they teach and for all purposes.

### BACKGROUND

The present disclosure relates to a sealing member for sealing a flow path of a medical tube so as to allow the flow path of the medical tube to be in fluid communication and a blood bag set.

A sealing member is used to allow a medical tube to be opened from a closed state into a fluid communication state. The sealing member is a cap-shaped member fitted in the lumen of the medical tube, and seals the medical tube in an initial state. The sealing member includes a breakable portion. In response to properly bending the sealing member during an opening operation, the breakable portion is broken and the sealing member allows the medical tube to be opened from the closed state into the fluid communication state.

Such a sealing member is used for, for example, a blood bag set as described in Japanese Patent Document No. 08-275986 A.

### BRIEF SUMMARY

Insufficient bending of the sealing member during the opening operation causes a poor fluid communication in which part of the breakable portion remains connected. Even in such a poor fluid communication arrangement, where the opening is defective, the sealing member can allow some liquid such as blood to flow and pass through the medical tube. However, in the case of a traditional sealing member, when blood passes through a narrow breakable portion or fractured region with a poor partially blocked opening, red blood cells may become damaged and cause hemolysis.

It is an object of the present disclosure to solve the above issues and other problems associated with conventional sealing members and medical tubes.

According to one aspect of the disclosure below, provided is a sealing member including: a base having an outer circumferential face for contact with an inner face of a medical tube, the base having a base flow path for allowing fluid to pass inside of the base, the base flow path extending in a direction of an axis of the sealing member, the base flow path being open at a proximal end of the base; a neck located at a distal end of the base such that the base has an outer diameter that reduces; a plug connected to the base through the neck, the plug extending from the base toward a side of location of a distal end of the sealing member in the direction of the axis of the sealing member, the plug occluding the base flow path; and a breakable portion including a thinnest portion of a boundary between a proximal end of the plug and the neck, in which, in viewing from the side of location of the distal end of the sealing member in the direction of the axis of the sealing member, the breakable portion is non-circular in shape, the breakable portion including a plurality of tops and a plurality of connections between the plurality of tops, and a distance between the base and the axis is larger than a radius of a circle inscribed in the plurality of connections, and the base is located outside the plurality of connections in a radial direction of the sealing member.

According to another aspect of the disclosure below, provided is a blood bag set including: the sealing member of the aspect described above; a blood bag that houses whole blood or a blood component; and a medical tube in communication with the blood bag, in which the sealing member occludes the medical tube in contact with an inner face of the medical tube so as to allow the medical tube to be in communication.

According to the sealing member and the blood bag set of the aspects described above, the breakable portion is broken smoothly, thereby reducing the occurrence of poor fluid communication. Further, according to the sealing member and the blood bag set, even when a defective opening operation occurs in the sealing member, hemolysis can be prevented because the breakable portion is large in flow-path cross-sectional area.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages are described herein and will be apparent to those skilled in the art upon consideration of the following Detailed Description and in view of the figures.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a side view of a sealing member according to at least one embodiment of the present disclosure.

FIG. 2 is a six-sided view of the sealing member of FIG. 1 in orthographic projection.

FIG. 3 is a longitudinal section view of the sealing member of FIG. 1.

FIG. 4A is a perspective view of the sealing member of FIG. 1.

FIG. 4B is a view of a broken portion of the sealing member after separation of a plug of the sealing member of FIG. 1 as viewed from the side of the location of the distal end of the sealing member in the axial direction of the sealing member.

FIG. 5 is a perspective view of the sealing member illustrating the distribution of stress generated in the breakable portion resulting from bending of the plug of the sealing member of FIG. 1.

FIG. 6 is a schematic view of the sealing member of FIG. 1 illustrating an opening failure condition.

FIG. 7 is a schematic view of a sealing member illustrating an opening failure condition according to a comparative example.

FIG. 8 is a chart including a comparison of the sealing member according to the comparative example and the sealing member of FIG. 1 in the cross-sectional shape and opening area of the breakable portion.

FIG. 9 is a schematic view of a blood bag set including the sealing member of FIG. 1.

FIG. 10A is a view of the shape of a breakable portion according to a first modification of at least one embodiment of the present disclosure.

FIG. 10B is a view of the shape of a breakable portion according to a second modification of at least one embodiment of the present disclosure.

FIG. 11 is a section view of the shape of a breakable portion according to a third modification of at least one embodiment of the present disclosure.

### DETAILED DESCRIPTION

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The ensuing description provides embodiments only, and is not intended to limit the scope, applicability, or configuration of the claims. Rather, the ensuing description will provide those skilled in the art with an enabling description for implementing the described embodiments. It being understood that various changes may be made in the function and arrangement of elements without departing from the spirit and scope of the appended claims.

Unless otherwise specified, all technical terms and scientific terms used herein have the same meaning that those skilled in the art commonly understand. All patents, applications and other publications and information cited herein are incorporated herein by reference in their entirety.

It is with respect to the above issues and other problems that the embodiments presented herein were contemplated.

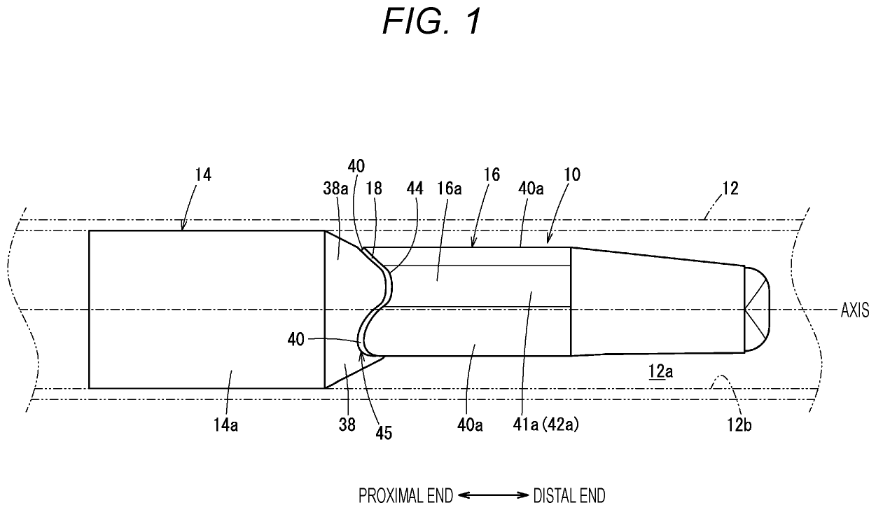

As illustrated in FIG. 1, a sealing member 10 according to the present disclosure is used for sealing a flow path 12a of a medical tube 12. The sealing member 10 is liquid-tightly secured to the flow path 12a of the medical tube 12 in the initial state (e.g., closed state). Stated another way, when the sealing member is disposed inside the lumen of the medical tube 12 in the initial state, fluid is prevented from moving past the sealing member in the flow path 12a. The sealing member 10 includes a base 14, a plug 16, and a breakable portion 18. In the initial state, the base 14 and the plug 16 are in connection through the breakable portion 18, and the plug 16 seals the flow path 12a. In response to bending the plug 16, the breakable portion 18 is broken, so that the sealing member 10 is in arranged in a fluid communication state and allows the flow path 12a to be in open fluid communication. In at least some embodiments, the breakable portion 18 may correspond to a joint disposed between the base 14 and the plug 16. The joint may define at least one controlled line, area, stress concentration groove, or other region along which a fracture may promulgate during bending. For instance, when a predetermined bending force is applied to a portion of the medical tube 12 (e.g., between the base and the plug 16), for example, during an opening operation, the plug 16 may be caused to fracture along the joint providing an at least partial separation between the plug 16 and the base 14. This at least partial separation allows fluid (e.g., blood, etc.) to flow past the sealing member 10 along the flow path 12a. The breakable portion 18 or joint is disposed between the base 14 and the plug 16. In at least some embodiments of the present disclosure, the at least one stress concentration groove extends into the sealing member 10 in a direction toward the axis of the sealing member 10. In this example, the at least one stress concentration groove extends around a periphery of the breakable portion 18 or joint.

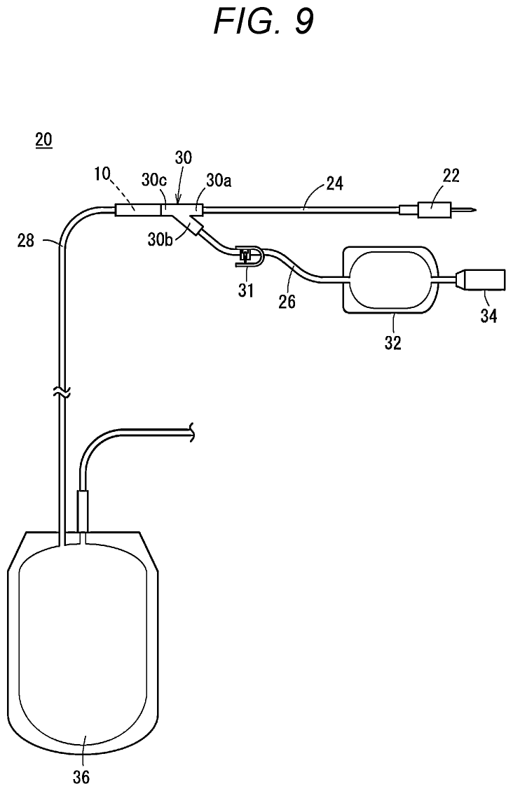

Such a sealing member 10 may be used for, for example, a blood bag set 20 illustrated in FIG. 9. In some embodiments, the blood bag set 20 may include a blood sampling needle 22, a first tube 24, a second tube 26, a third tube 28, a branch portion 30, an initial flow blood bag 32, a sampling holder 34, and a blood bag 36.

The blood sampling needle 22 is connected to one end of the first tube 24. The other end of the first tube 24 is connected to a first port 30a of the branch portion 30 branched in three directions. As illustrated in FIG. 9, the second tube 26 is connected to a second port 30b of the branch portion 30, and the third tube 28 is connected to a third port 30c of the branch portion 30. The second tube 26 connects the branch portion 30 and the initial flow blood bag 32. The sampling holder 34 is connected downstream of the initial flow blood bag 32. The third tube 28 connects the branch portion 30 and the blood bag 36 (e.g., blood sampling bag, etc.). The sealing member 10 of FIG. 1 is attached to the third tube 28 near the third port 30c of the branch portion 30. In the initial state, the sealing member 10 occludes the third port 30c and the third tube 28.

The blood sampling needle 22 of the blood bag set 20 draws blood from a donor (e.g., a blood donor, etc.). Because the third port 30c of the branch portion 30 is occluded by the sealing member 10 in the initial state, the drawn blood flows into the second tube 26 through the second port 30b. The blood is stored in the initial flow blood bag 32. The sampling holder 34 is used to collect a portion of the drawn blood in, for example, a sample tube.

The operation of collecting the blood of the donor into the blood bag 36 is performed by occluding the second tube 26 of the blood bag set 20 with a clamp 31 and allowing the sealing member 10 to be in communication. In such a manner, the sealing member 10 is used, for example, for allowing a flow path of the blood bag set 20 to be in communication and occluding the flow path thereof.

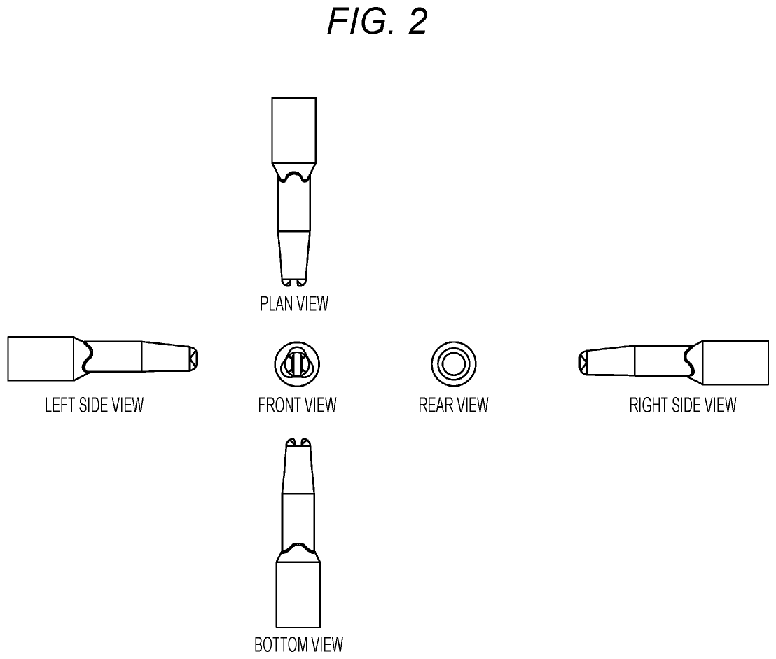

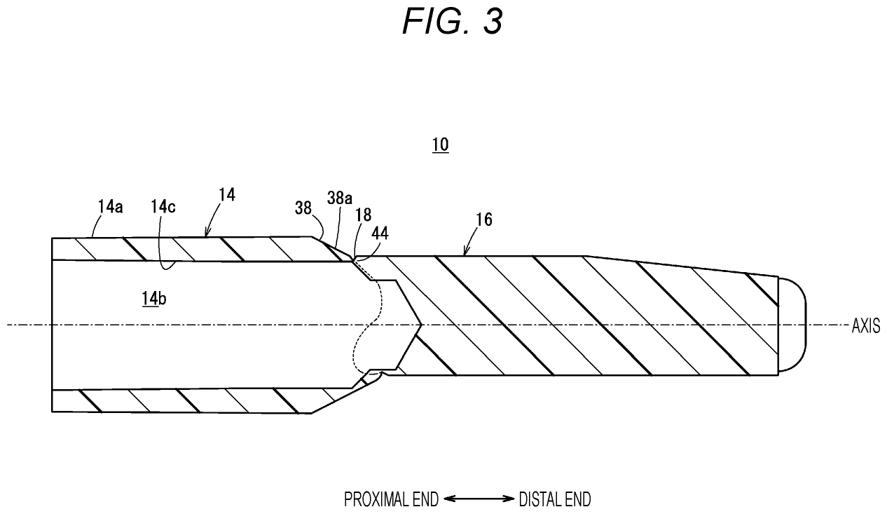

As illustrated in FIGS. 1 to 4A, the sealing member 10 includes the base 14 and the plug 16. According to at least one embodiment of the present disclosure, the base 14 has a cylindrical shape extending in the axial direction of the sealing member 10 (e.g., from a proximal end of the base 14 to a distal end of the base 14). An outer circumferential face 14a of the base 14 is secured to the flow path 12a in liquid-tight contact with an inner circumferential face 12b of the flow path 12a of the medical tube 12. The medical tube 12 is, for example, the third tube 28. As illustrated in FIG. 3, the base 14 of the sealing member 10 has a base flow path 14b inside of the base 14. The base flow path 14b forms a lumen that is surrounded by the inner circumferential face 14c of the base 14 concentric with the outer circumferential face 14a thereof. The base flow path 14b extends in the axial direction of the sealing member 10. The proximal end of the base flow path 14b is open at the proximal end of the base 14. The distal end of the base flow path 14b extends to the inside of the plug 16.

As illustrated in FIG. 1, the base 14 has a neck 38 arranged at the distal end of the base 14. The neck 38 is an extending portion that reduces in outer diameter to the distal end of the base 14 such that the outer diameter of the neck 38 is smaller than the outer diameter of the base 14 (e.g., at the proximal end of the base 14). The neck 38 has an outer circumferential face 38a including a conical face.

The plug 16 extends from the neck 38 toward its distal end in the axial direction of the sealing member 10. In the initial (or closed) state, the plug 16 is integrally connected to the base 14 via the neck 38. The plug 16 is interconnected to the base 14 at the distal end of the base 14. As illustrated in FIG. 3, the plug 16 has a solid portion. The solid portion of the plug 16 covers the side of location of the distal end of the base flow path 14b such that the plug 16 occludes the distal end of the base flow path 14b. As illustrated in FIG. 1, the plug 16 has an outer diameter smaller than that of the base 14. Therefore, when the sealing member 10 is secured to the medical tube 12, the plug 16 is separated from the inner circumferential face 12b of the medical tube 12. Stated another way, when the sealing member 10 is arranged inside the medical tube 12 in the initial state, and the outer circumferential face 14a of the base 14 is disposed in direct contact with the inner circumferential face 12b of the medical tube 12, the plug 16 is arranged offset from the inner circumferential face 12b of the medical tube 12 (e.g., providing a space between the outer face of the plug 16 and the inner circumferential face 12b of the medical tube 12). In the initial or closed state, the plug 16 completely covers and blocks the base flow path 14b lumen at the distal end of the base 14. Additionally or alternatively, in the initial (or closed) state, the at least one stress concentration groove of the breakable portion 18, or joint, is entirely unbroken along the periphery of the breakable portion 18. In a fluid communication state, the at least one stress concentration groove of the breakable portion 18 is broken along at least a portion of the periphery of the breakable portion 18.

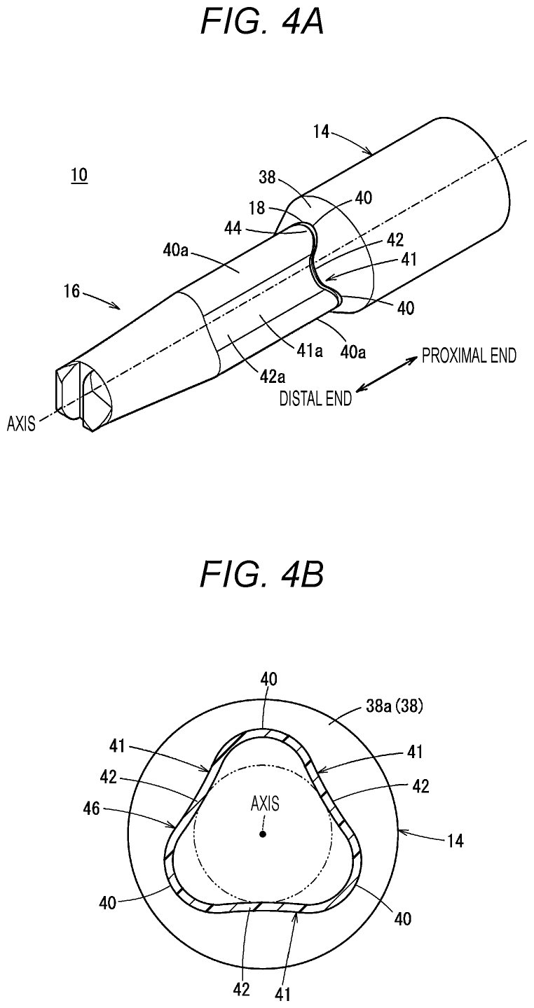

As illustrated in FIG. 4A, the plug 16 has three ridges 40a radially protruding outside from the central axis (AXIS) of the sealing member 10. The three ridges 40a of the plug 16 are each circumferentially disposed apart from one another at an angle of 120° around the central axis. Each of the ridges 40a extends linearly in the axial direction of the sealing member 10. The breakable portion 18 includes each top 40 at the boundary between the corresponding ridge 40a and the neck 38. A relatively flat curved face 41a is located between one ridge 40a and another ridge 40a adjacent thereto. The curved face 41a is a portion having a relatively small curvature, and connects two adjacent tops 40 due to such a smooth curved face. The breakable portion 18 includes a connection 41 at the boundary between the curved face 41a and the neck 38. In some examples, at least one of the plug 16 and the breakable portion 18 or joint comprises a noncircular cross-sectional shape, the noncircular cross-sectional shape surrounds the axis of the sealing member 10. This noncircular cross-sectional shape may undulate between concave and convex portions of the cross-sectional shape following an outer peripheral surface of the plug 16. In one example, the noncircular cross-sectional shape undulates longitudinally (e.g., in a proximal and distal direction) as well as circumferentially as the noncircular cross-sectional shape follows the outer peripheral surface of the plug 16.

As illustrated, the curved face 41a of the plug 16 may have a recessed face portion 42a curved in a recessed shape toward the central axis of the sealing member 10. In some embodiments, the recessed face portion 42a may correspond to a concave outer surface portion of the plug 16 (e.g., having a central portion extending inwardly toward the central axis of the sealing member 10) that is arranged between and connecting two adjacent ridges 40a. The recessed face portion 42a includes a curved face having a center of curvature located outside the plug 16. The breakable portion 18 includes each recess 42 at the boundary between the corresponding recessed face portion 42a and the neck 38. The connection 41 including the recess 42 is a portion where the breakable portion 18 is relatively easily broken by the force of bending the plug 16. For instance, the breakable portion 18 may correspond to a joint having a scored or otherwise relieved cross-sectional area that allows a bending force to fracture along the joint and provide an opening between the base 14 and the plug 16. In the present embodiment, such an easily breakable portion appears in three directions, and thus a user can break the breakable portion 18 without concern for the orientation of the plug 16.

As illustrated in FIG. 1, the breakable portion 18 is located at the boundary between the proximal end of the plug 16 and the neck 38. The breakable portion 18 includes a groove 44 along a line where an outer circumferential face 16a of the plug 16 and an outer circumferential face 38a of the neck 38 intersect mutually. In the present embodiment, the groove 44 is the thinnest portion 45 that is the thinnest portion of the sealing member 10. The groove 44 extends linearly and extends circumferentially along the outer circumferential face 38a of the neck 38. As illustrated in FIGS. 4A and 4B, when from the side of the location of the distal end of the sealing member 10 in the axial direction of the sealing member 10, the groove 44 has a plurality of such tops 40 and such recesses 42 (connections 41) as described above that are located between the tops 40, and extends circumferentially in a wavy or undulating manner. As illustrated in FIG. 1, the groove 44 is also serpentine in the axial direction of the sealing member 10 in side view. In such a manner, the groove 44 extends three-dimensionally without extending along one plane.

As illustrated in FIG. 3, the groove 44 of the breakable portion 18 is the thinnest of the other portions of the sealing member 10. Thus, the groove 44 can be easily broken resulting from the communicating or opening operation of bending the plug 16. In response to the communicating or opening operation, the entire region of the groove 44 is broken and the plug 16 is separated from the base 14. The base 14 with the breakable portion 18 broken has a broken portion 46 illustrated in FIG. 4B. As illustrated, the groove 44 has the broken portion 46 circumferentially wavy in viewing from the side of location of the distal end of the sealing member 10 in the axial direction of the sealing member 10. Among other things, FIG. 4B shows a circle (in dashed lines) inscribed within the noncircular cross-sectional shape. The circle is shown contacting a portion of the joint or breakable portion 18 adjacent each of the recessed face portions 42. A radially extending space is shown disposed between the circle inscribed within the noncircular cross-sectional shape and a portion of the joint adjacent each of the tops 40 of the ridges 40a.

As illustrated in FIG. 1, because the plug 16 of the sealing member 10 is separated (e.g., offset a distance, etc.) from the inner circumferential face 12b of the medical tube 12 in the initial state, the plug 16 can be bent within the medical tube. In response to the application of a predetermined bending force between the plug 16 and the base 14, for example, from the outside of the medical tube 12, the breakable portion 18 is broken. Although the usage is not limited, the recessed face portion 42a between each ridge 40a of the plug 16 is recessed, and thus the plug 16 is a portion to which the user can easily input or apply the opening operation force.

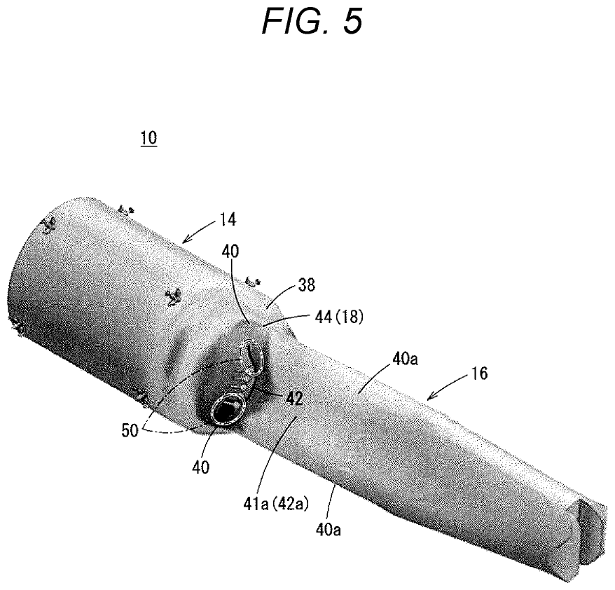

As illustrated in FIG. 5, in response to the input or application of the force from the recess 42 toward the central axis, a stress concentration portion 50 is generated at one end and the other end in the circumferential direction of the recess 42. Such stress concentration portions 50 as described above are each a start point of breakage of the breakable portion 18 resulting from input of a relatively weak bending force. Due to expansion of the breaking part of the breakable portion 18, the breakable portion 18 of the sealing member 10 is reliably broken even with input of a weak bending force. As a result, the sealing member 10 can lower the frequency of failed opening operations and poor fluid communication.

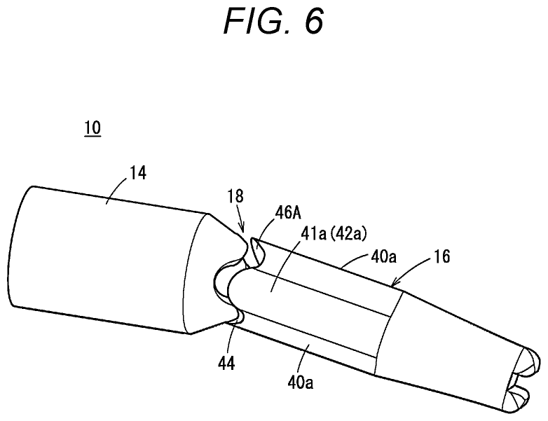

As illustrated in FIG. 6, even in a case where part of the breakable portion 18 remains without being completely broken, the sealing member 10 has a three-dimensionally wavy broken portion 46A. In such a sealing member 10, the gap between the neck 38 and the plug 16 along the broken portion 46A serves as a fluid flow path. The three-dimensionally wavy broken portion 46A can have a relatively large flow-path cross-sectional area. Therefore, even when a defective opening operation is performed, the sealing member 10 of the present disclosure can prevent the red blood cells in the blood from being damaged, resulting in reduction of the risk of hemolysis.

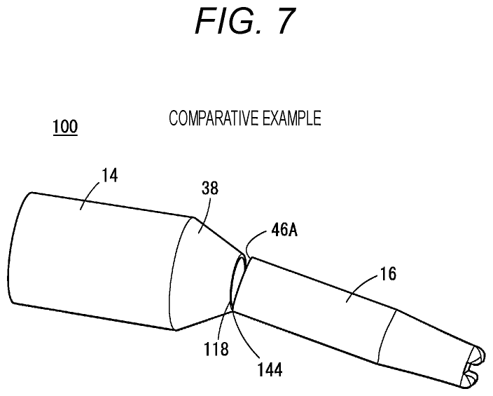

In comparison with the sealing member 10 of the present disclosure, a sealing member 100 of a comparative example, shown in FIG. 7, includes a breakable portion 118 including a circular groove 144. In the sealing member 100 of the comparative example, a flow path formed by a broken portion 46A when a defective opening operation is performed is narrower providing poor fluid communication.

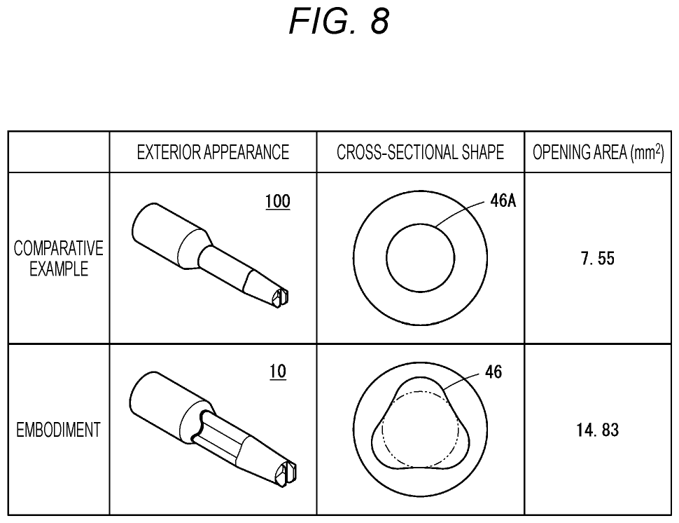

As illustrated in FIG. 8, the sealing member 100 of the comparative example has an opening area of 7.55 mm², whereas the sealing member 10 of the present embodiment has an opening area of 14.83 mm². As can be appreciated, the opening area of the sealing member 10 of the present disclosure is larger than the opening area of the sealing member 100 of the comparative example. In the sealing member 10, the cross-sectional area of the flow path formed when a defective opening operation is performed increases in accordance with the length of the broken portion 46, and in the sealing member 100 of the comparative example, the cross-sectional area of the flow path formed when a defective opening operation is performed increases in accordance with the length of the broken portion 46A. However, the broken portion 46 of the sealing member 10 of the present embodiment is longer than the broken portion 46A of the sealing member 100 of the comparative example. Therefore, even when a defective opening operation is performed, the sealing member 10 of the present disclosure provides a larger flow-path cross-sectional area formed by the broken portion 46, resulting in better flow throughput and a reduction of the risk of hemolysis.

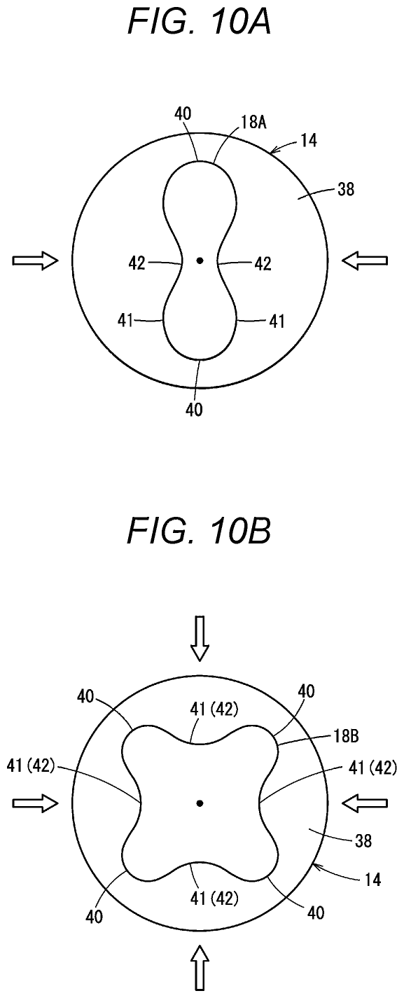

As provided above, a sealing member 10 in which the groove 44 of the breakable portion 18 includes the three tops 40 has been shown and described, but the present disclosure is not limited thereto. For example, in a first modification, as illustrated in FIG. 10A, a breakable portion 18A may include two tops 40 when viewed from the side of the location of the distal end of a sealing member 10 in the axial direction of a sealing member 10. Additionally or alternatively, in a second modification, as illustrated in FIG. 10B, a breakable portion 18B may include four tops 40 when viewed from the side of the location of the distal end of a sealing member 10 in the axial direction of a sealing member 10. The description provided above for the features of the sealing member 10 having the shape illustrated in FIG. 4B may also apply to the features of the sealing member 10 having the shapes illustrated in FIG. 10A and/or FIG. 10B.

As described above, the thinnest portion 45, including the groove 44 (e.g., as shown in FIG. 3), comprises a V-shaped cross-section on the outer circumference side of the sealing member 10. However, the present embodiment is not limited thereto. In a third modification, another configuration example of the thinnest portion 45 will be described.

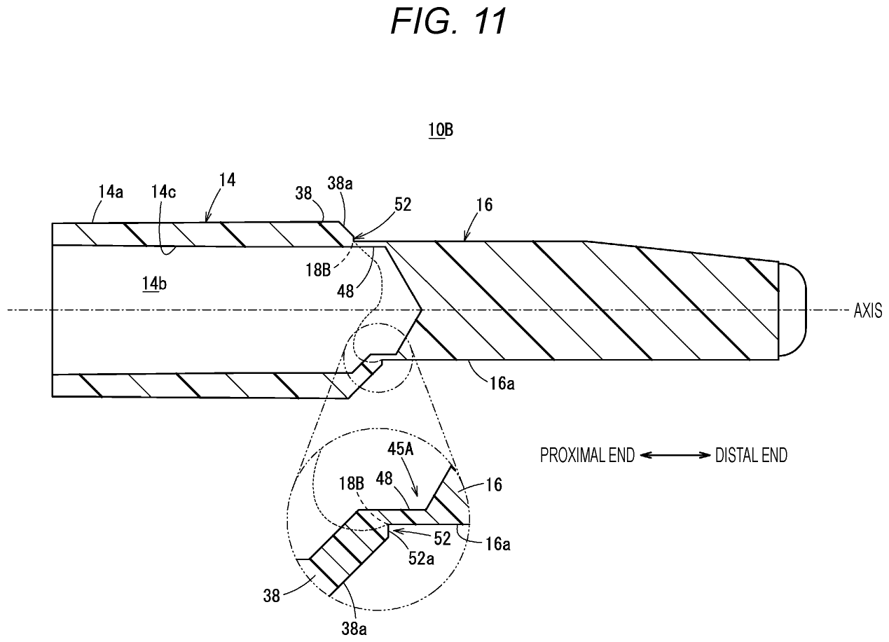

As illustrated in FIG. 11, a sealing member 10B of the present disclosure may include a thinnest portion 45A. The thinnest portion 45A includes an inner thin portion 48 that increases in diameter of a plug 16 from the inner circumference side, and a step 52 provided at the boundary between a neck 38 and the plug 16. The step 52 is located at the boundary between an outer circumferential face 38a of the neck 38 and an outer circumferential face 16a of the plug 16. The step 52 includes a step face 52a facing a direction perpendicular to the axial direction of the sealing member 10B. The step face 52a and the outer circumferential face 16a of the plug 16 intersect substantially perpendicularly with each other. On the step 52, the neck 38 reduces in thickness and the thinnest portion 45A is located. The thinnest portion 45A is located between the outer circumferential face 16a and the inner thin portion 48, and has a width in the axial direction of the sealing member 10B. The thinnest portion 45A extends over the entire region in the circumferential direction of the plug 16.

A breakable portion 18B of the third modification, illustrated in FIG. 11, is provided on a step 52 of the thinnest portion 45A. The breakable portion 18B extends over the entire region in the circumferential direction. In response to the application of a bending force to the plug 16, stress concentrates on the step 52 of the thinnest portion 45A. As a result, the breakable portion 18B is broken. In such a manner, in the sealing member 10B of the present modification, the breakable portion 18B of the thinnest portion 45A can be broken resulting from a bending operation on the plug 16.

The sealing member 10 and the blood bag set 20 of the present embodiment are summarized as follows.

According to the embodiments described above, provided is a sealing member 10 including: a base 14 having an outer circumferential face 14a for contact with an inner face of a medical tube 12, the base 14 having a base flow path 14b for allowing fluid to pass inside of the base 14, the base flow path extending in a direction of an axis of the sealing member 10, the base flow path being open at a proximal end of the base 14; a neck 38 located at a distal end of the base 14 such that the base 14 has an outer diameter that reduces; a plug 16 connected to the base 14 through the neck 38, the plug 16 extending from the base 14 toward a side of location of a distal end of the sealing member 10 in the direction of the axis of the sealing member 10, the plug 16 occluding the base flow path; and a breakable portion 18 including a thinnest portion 45 of a boundary between a proximal end of the plug 16 and the neck 38, in which, in viewing from the side of location of the distal end of the sealing member 10 in the direction of the axis of the sealing member 10, the breakable portion 18 is non-circular in shape, the breakable portion 18 including a plurality of tops 40 and a plurality of connections 41 between the plurality of tops 40, a distance between the base 14 and the axis is larger than a radius of a circle inscribed in the plurality of connections 41, and the base 14 is located outside the plurality of connections 41 in a radial direction of the sealing member 10. Stated another way, a first radial distance from the axis to the outer peripheral surface of the plug 16 at each ridge 40a is greater than a second radial distance from the axis to the outer peripheral surface of the plug 16 at each recessed face portion 42a.

In response to bending of the plug, the sealing member described above has a stress concentration portion 50 on the breakable portion. Thus, the breakable portion can be broken reliably even with a weak force. As a result, the frequency of occurrence of a defective opening operation and poor fluid communication of the breakable portion is inhibited. Further, the breakable portion has a large opening area of the gap formed by the breakable portion, even in the occurrence of a defective opening operation with the breakable portion not partially broken. Therefore, the sealing member can reduce the risk of hemolysis.

In the sealing member described above, the breakable portion may include the thinnest portion extending in a circumferential direction over entire of the boundary between the proximal end of the plug and the neck along the boundary. The breakable portion can be broken with a force from any direction in the circumferential direction, and thus the sealing member provides excellent reliability and operability.

When viewed from the side of the location of the distal end of the sealing member in the direction of the axis of the sealing member, each of the plurality of connections of the breakable portion may include a recess 42 recessed toward a center. A portion where the plug is easily bent is located in a relatively flat portion between two adjacent tops of the plurality of tops. Therefore, the breakable portion can be broken more reliably.

When viewed from the side of the location of the distal end of the sealing member in the direction of the axis of the sealing member, the breakable portion may be wavy in shape in the circumferential direction due to the plurality of tops and the recess. Due to the wavy shape of the breakable portion, the opening area increases when a defective or failed opening operation occurs.

When viewed from the side of the location of the distal end of the sealing member in the direction of the axis of the sealing member, the plurality of tops of the breakable portion may be three in number. A portion where the plug is easily bent is located in a relatively flat portion between two adjacent tops of the plurality of tops. In this case, because the direction in which the plug is easily bent appears in three directions, the user can perform a bending operation without being conscious of the orientation of the plug. Further, because the relatively flat portion is large in width, the breakable portion can be easily broken even with a weak pressurizing force.

In the sealing member described above, the neck may have a conical face inclined such that the diameter of the neck gradually reduces toward the side of location of the distal end of the sealing member in the direction of the axis of the sealing member, and the breakable portion may be located on the conical face. Such a sealing member can include a breakable portion that is three-dimensionally wavy or undulated in shape at the boundary between the neck and the plug, and thus the opening area of the flow path can increase even in when a defective or failed opening operation is performed. Therefore, the sealing member can reduce the risk of hemolysis when a defective or failed opening operation is performed.

According to the embodiments described above, provided is a blood bag set 20 including: the sealing member described above; a blood bag 36 that houses whole blood or a blood component; and a medical tube in communication with the blood bag 36, in which the sealing member occludes the medical tube in contact with an inner face of the medical tube so as to allow the medical tube to be in communication.

The blood bag set can reduce the risk of hemolysis with the sealing member.

Note that the present disclosure is not limited to the embodiments described above, and thus various configurations may be adopted without departing from the gist of the present disclosure.

References in the specification to “one embodiment,” “an embodiment,” “an example embodiment,” “some embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in conjunction with one embodiment, it is submitted that the description of such feature, structure, or characteristic may apply to any other embodiment unless so stated and/or except as will be readily apparent to one skilled in the art from the description. The present disclosure, in various embodiments, configurations, and aspects, includes components, methods, processes, systems and/or apparatus substantially as depicted and described herein, including various embodiments, subcombinations, and subsets thereof. Those of skill in the art will understand how to make and use the systems and methods disclosed herein after understanding the present disclosure. The present disclosure, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease, and/or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments, configurations, or aspects for the purpose of streamlining the disclosure. The features of the embodiments, configurations, or aspects of the disclosure may be combined in alternate embodiments, configurations, or aspects other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the disclosure.

Moreover, though the description of the disclosure has included description of one or more embodiments, configurations, or aspects and certain variations and modifications, other variations, combinations, and modifications are within the scope of the disclosure, e.g., as may be within the skill and knowledge of those in the art, after understanding the present disclosure. It is intended to obtain rights, which include alternative embodiments, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

It is to be appreciated that any feature described herein can be claimed in combination with any other feature(s) as described herein, regardless of whether the features come from the same described embodiment.

As used herein, the singular forms “a,” “an,” and “the” are intended to include the plural forms as well, unless the context clearly indicates otherwise. It will be further understood that the terms “include,” “including,” “includes,” “comprise,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. The term “and/or” includes any and all combinations of one or more of the associated listed items.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B, and C together. When each one of A, B, and C in the above expressions refers to an element, such as X, Y, and Z, or a class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and this disclosure.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

What is claimed is:

1. A sealing member comprising:
a base having an outer circumferential face for contact with an inner face of a medical tube, the base having a base flow path for allowing fluid to pass inside of the base, the base flow path extending in a direction of an axis of the sealing member, the base flow path being open at a proximal end of the base;
a neck located at a distal end of the base such that the base has an outer diameter that reduces;
a plug connected to the base through the neck, the plug extending from the base toward a side of location of a distal end of the sealing member in the direction of the axis of the sealing member, the plug occluding the base flow path; and
a breakable portion including a thinnest portion of a boundary between a proximal end of the plug and the neck, wherein
in viewing from the side of location of the distal end of the sealing member in the direction of the axis of the sealing member, the breakable portion is non-circular in shape, the breakable portion including a plurality of tops and a plurality of connections between the plurality of tops, and
a distance between the base and the axis is larger than a radius of a circle inscribed in the plurality of connections, and the base is located outside the plurality of connections in a radial direction of the sealing member.

2. The sealing member according to claim 1, wherein the breakable portion includes the thinnest portion extending in a circumferential direction over an entire of the boundary between the proximal end of the plug and the neck along the boundary.

3. The sealing member according to claim 2, wherein, in viewing from the side of location of the distal end of the sealing member in the direction of the axis of the sealing member, each of the plurality of connections of the breakable portion includes a recess recessed toward a center.

4. The sealing member according to claim 3, wherein, in viewing from the side of location of the distal end of the sealing member in the direction of the axis of the sealing member, the breakable portion is wavy in shape in the circumferential direction due to the plurality of tops and the recess.

5. The sealing member according to claim 2, wherein, in viewing from the side of location of the distal end of the sealing member in the direction of the axis of the sealing member, the plurality of tops of the breakable portion is three in number.

6. The sealing member according to claim 1, wherein the neck has a conical face inclined such that a diameter of the neck gradually reduces toward the side of location of the distal end of the sealing member in the direction of the axis of the sealing member, and the breakable portion is located on the conical face.

7. A blood bag set comprising:
a sealing member comprising:
a base having an outer circumferential face for contact with an inner face of a medical tube, the base having a base flow path for allowing fluid to pass inside of the base, the base flow path extending in a direction of an axis of the sealing member, the base flow path being open at a proximal end of the base;
a neck located at a distal end of the base such that the base has an outer diameter that reduces;
a plug connected to the base through the neck, the plug extending from the base toward a side of location of a distal end of the sealing member in the direction of the axis of the sealing member, the plug occluding the base flow path; and
a breakable portion including a thinnest portion of a boundary between a proximal end of the plug and the neck, wherein
in viewing from the side of location of the distal end of the sealing member in the direction of the axis of the sealing member, the breakable portion is non-circular in shape, the breakable portion including a plurality of tops and a plurality of connections between the plurality of tops, and
a distance between the base and the axis is larger than a radius of a circle inscribed in the plurality of connections, and the base is located outside the plurality of connections in a radial direction of the sealing member;
a blood bag that houses whole blood or a blood component; and
a medical tube in communication with the blood bag, wherein
the sealing member occludes the medical tube in contact with an inner face of the medical tube so as to allow the medical tube to be in communication.

8. A sealing member for a medical tube, the sealing member comprising:
a base extending along an axis of the sealing member from a proximal end of the base to a distal end of the base, wherein the base comprises a base flow path lumen extending from the proximal end of the base to the distal end of the base;
a plug interconnected to the base at the distal end of the base, wherein the plug completely covers and blocks the base flow path lumen at the distal end of the base; and
a joint disposed between the base and the plug, the joint comprising at least one stress concentration groove extending into the sealing member in a direction toward the axis of the sealing member, the at least one stress concentration groove extending around a periphery of the joint;
wherein at least one of the plug and the joint comprises a noncircular cross-sectional shape, the noncircular cross-sectional shape surrounding the axis of the sealing member, and wherein the noncircular cross-sectional shape undulates between concave and convex portions following an outer peripheral surface of the plug.

9. The sealing member according to claim 8, wherein the noncircular cross-sectional shape undulates longitudinally as well as circumferentially as the noncircular cross-sectional shape follows the outer peripheral surface of the plug.

10. The sealing member according to claim 8, wherein the sealing member is movable between a closed state and a fluid communication state, wherein in the closed state the at least one stress concentration groove is entirely unbroken along the periphery of the joint, and wherein in the fluid communication state the at least one stress concentration groove is broken along at least a portion of the periphery of the joint.

11. The sealing member according to claim 10, wherein the noncircular cross-sectional shape of the plug comprises at least two recessed face portions and at least two ridges, wherein a first recessed face portion of the at least two recessed face portions is disposed between adjacent ridges of the at least two ridges.

12. The sealing member according to claim 11, wherein the at least two ridges comprise convex surfaces of the outer peripheral surface of the plug, wherein the at least two recessed face portions comprise concave surfaces of the outer peripheral surface of the plug.

13. The sealing member according to claim 12, wherein a first radial distance from the axis to the outer peripheral surface of the plug at each of the at least two ridges is greater than a second radial distance from the axis to the outer peripheral surface of the plug at each of the at least two recessed face portions.

14. The sealing member according to claim 12, wherein a circle inscribed within the noncircular cross-sectional shape contacts a portion of the joint adjacent each of the at least two recessed face portions, and wherein a radially extending space is disposed between the circle inscribed within the noncircular cross-sectional shape and a portion of the joint adjacent each of the at least two ridges the joint.

15. The sealing member according to claim 12, wherein the at least one stress concentration groove extending around the periphery of the joint comprises a thinnest section of material of the sealing member.

16. The sealing member according to claim 12, wherein the base comprises an outer circumferential face arranged adjacent the proximal end of the base, and wherein the outer circumferential face is sized to seal against an inner face of a medical tube in a liquid-tight contact arrangement.

17. The sealing member according to claim 16, wherein, in the closed state, the sealing member prevents fluid flow past the sealing member through the medical tube, and wherein, in the fluid communication state, the sealing member allows fluid flow past the sealing member through the medical tube via the base flow path lumen.

18. The sealing member according to claim 16, wherein the distal end of the base tapers from a first outer circumferential face diameter to a second outer circumferential face diameter, wherein the first outer circumferential face diameter is greater than the second outer circumferential face diameter.

19. The sealing member according to claim 8, wherein the noncircular cross-sectional shape of the plug comprises three recessed face portions and three ridges, wherein a first recessed face portion of the three recessed face portions is disposed between a first set of two adjacent ridges of the three ridges, wherein a second recessed face portion of the three recessed face portions is disposed between a second set of two adjacent ridges of the three ridges, and wherein a third recessed face portion of the three recessed face portions is disposed between a third set of two adjacent ridges of the three ridges.

20. The sealing member according to claim 19, wherein each ridge of the three ridges is separated from one another by 120 degrees.
